(19) United States
(12) Patent Application Publication
(10) Pub. No.: US 2010/0136128 A1
(43) Pub. Date: Jun. 3, 2010

(54) Odor control fragrance additive

(76) Inventors: Marni Markell Hurwitz, Far Hills, NJ (US); Ernest D. Buff, Far Hills, NJ (US)

Correspondence Address: ERNEST D. BUFF;ERNEST D. BUFF AND ASSOCIATES, LLC., 231 SOMERVILLE ROAD, BEDMINSTER, NJ, 07921, US

(21) Appl. No.: 12/590,420

(22) Filed: Nov. 6, 2009

Related U.S. Application Data
(63) Continuation-in-part of application No. 12/583,690, filed on Aug. 24, 2009.
Application No. 12/583,690 is a continuation-in-part of application No. 11/408,493, filed on Apr. 21, 2006, now Pat. No. 7,549,396.
Application No. 11/408,493 is a continuation-in-part of application No. 11/348,723, filed on Feb. 7, 2006, now Pat. No. 7,637,229.
(60) Provisional application No. 60/650,795, filed on Feb. 8, 2005.

(51) Int. Cl. A61L 9/014; B29C 43/00; A61L 9/012; A61K 9/14; A01K 29/00

(52) U.S. Cl. 424/499; 264/122; 424/76.1; 424/76.6; 424/76.9; 119/172

(57) ABSTRACT

An odor control fragrant additive comprises compacted pellets or granules infiltrated with fragrant oil. Cellulosic material, activated carbon and binder are mixed together and compacted to produce compacted pellets or granules. The cellulosic material preferably is peat moss, which swells up during absorption of fragrant oil and acts as fragrant oil storage. Fragrant oil is delivered to activated carbon that has large surface area, which evaporates the fragrance oil to release steady fragrance output. Thus, the activated carbon, which evaporates fragrance provides steady release of fragrance in the surrounding environment of fragrant compacted pellets or granules for a period of several months without overpowering the environment with excess fragrance. The fragrant compacted pellets or granules may be used to suppress malodor from trashcans or litter boxes and may provide stand-alone additions to drawers and cabinets, emitting pleasant fragrance.

### CROSS-REFERENCE TO RELATED APPLICATIONS

This application is a continuation-in-part of U.S. application Ser. No. 12/583,690, filed Aug. 24, 2009 which, in turn, is a continuation-in-part of U.S. application Ser. No. 11/408,493, filed Apr. 21, 2006 which, in turn, is a continuation-in-part of U.S. application Ser. No. 11/348,723, filed Feb. 7, 2006 which, in turn, claims the benefit of provisional Application 60,650,795, filed Feb. 8, 2005.

### BACKGROUND OF THE INVENTION

1. Field of the Invention

The present invention relates to odor control fragrance additives in the form of fragrant compacted pellets or granules. The fragrant compacted pellets or granules may be used within a trashcan masking malodor, or used as additive for odor control of for animal litter. When used with cat litter, the additive releases an odor controlling or odor masking substance when the animal uses a litter-box. The fragrant compacted pellets or granules may be used as stand-alone pellets or granules which, when placed in drawers or cabinets and the like release a pleasant fragrance for prolonged periods without releasing an overpowering fragrance.

2. Description of the Prior Art

Many commercial odor control products contain scented powdery material. For example, a baby powder is a talc powder that is scented with a fragrance. A commercial cat litter is a clay granular product scented with an overpowering scent or odor control fragrance. Litter products contain a scent that either over powers the litter smell or simply has ingredients that mask the litter odor.

Trashcan odor is highly objectionable and many trashcans are coated with odor masking fragrance. These polymeric trashcan liners have all the odor control ingredients concentrated only near the surface of the polymeric trash can liner and these ingredients do very little to control the odor of trash located in the central portion of the trashcan liner, away from the polymeric trashcan liner surface.

The litter odor is highly objectionable, especially in heated or air-conditioned closed rooms. In these products, the scent producing ingredient is incorporated within the cat litter and the scent from the scented litter is released all the time, creating over powering smell in the closed room. This overpowering scent is the reason why many people prefer unscented litter and hope to clean the litter box promptly when the litter box is used by the animal. To aid this cleaning process litter compositions have been developed that clump when the litter box is used for urination, thereby enabling the prompt easy cleaning of agglomerated clumps. Yet, the unscented litter progressively accumulates malodor and has to be replaced periodically.

Many patents disclose porous pellets made from polymeric foam or binder boned cellulose based materials and carrying fragrances. These fragrant pellets may be stand-alone pellets or a part of a cat litter composition. A number of prior art patents relate to micro encapsulation of fragrances and these fragrances are continually released. Some of the patents disclose encapsulation wherein the fragrance is prevented from slow release by having an impervious cell wall.

U.S. Pat. No. 5,019,434 to Matsumoto discloses a molded slow-release air freshener. The molded slow-release air freshener is prepared by having a liquid perfume composition absorbed into a porous molded product. The porous molded product is obtained by partially melt-bonding the particles of an ethylenevinyl acetate copolymer powder. The porous molded product has continuous gas-permeable pores and a bulk density in the range of 0.4 to 0.8 g/cm³, and the slow-release air freshener has an increased apparent volume of at least 1.2 times as large as its apparent volume before the absorption of the liquid perfume composition. The molded slow-release air freshener does not have cellulosic material that absorbs fragrance.

U.S. Pat. No. 6,213,409 to Warren, et al. discloses a time release fragrance sachet, method of using same and method of fabricating same. The time release fragrance sachet is for air freshening, particularly for use in clothing storage cabinets. The sachets comprise a container fabricated from a substance that is either sufficiently porous for passing perfumes or has openings capable of permitting fragrances to pass through the walls. The sachets contain thermoplastic particles, which have interstices containing fragrance materials. The polymer particles can be foamed particles produced using chemical blowing agents or direct gas extrusion processes. The thermoplastic particles do not contain cellulosic material.

U.S. Pat. No. 4,621,011 to Fleischer, et al. discloses agglomerated cellulosic particles. The cellulosic particle is useful as a cat litter and is manufactured by agglomerating a fibrous cellulosic feed material in the presence of water, compacting the surface of the agglomerated particles, and drying the particles. The particles are said to have a small amount of fragrance from about 0.01 weight percent to about 2.0 percent of the particle composition.

U.S. Pat. No. 5,970,916 to Yoder, et al. discloses clumping cellulosic animal litter. This clumping cellulosic animal litter comprises a plurality of cellulosic particles having a first coating of xanthan gum and a second coating of guar gum. The particles have a third coating of a tackiness-enhancing agent selected from of carboxymethylcellulose (CMC), methylcellulose, tapioca starch, xanthan gum, guar gum, guar gum derivatives, and karaya gum. The cellulosic particles have a small amount of fragrance in the range of from about 0.01 weight percent to about 2.0 percent of the particle composition.

U.S. Pat. No. 6,562,769 to Paatz, et al. discloses a method of producing aromatic beads. The method for producing aromatic shaped bodies, especially aromatic beads, with bulk densities greater than 700 g/l, uses a solid and essentially water-free premix. The premix has 65 to 95 wt. % carriers, 0 to 10 wt. % auxiliary agents, and 5 to 25 wt. % perfume and is subjected to granulation or compacted agglomeration. The agglomerated shaped beads scent washing and cleaning detergents in a wash machine. The beads do not contain cellulosic material and are not indicated to be for use as free standing perfume beads or in a trash bin or in combination with cat litter.

U.S. Pat. No. 6,276,300 to Lewis II et al. discloses animal litter. This litter composition includes paper, sawdust, and zeolite molecular sieve. The composition is formed into pellets or discs having a size, soil-like consistency, and texture that is attractive to dogs. In addition, the litter has a high degree of absorption and odor control. The litter composition may further include sphagnum moss to provide additional absorbency. The animal litter only has a small concentration of fragrance which is in the range of 0.05 to 0.3 weight percent.

U.S. Pat. No. 6,369,290 to Glaug, et al. discloses a time release odor control composition for a disposable absorbent article. This disposable absorbent article is provided with an odor control powder which is unscented in a dry state and releases a burst of fragrance when wetted, such as by human waste. The powder contains a relatively small amount of fragrance oil, such as 0.5% to 4% by weight, to prevent skin irritation to the wearer. The small amount of fragrance oil is microencapsulated in a starch, which constitutes from about 50% to 90%, and preferably about 70%, of the total weight of the particles. Sodium bicarbonate is also included in the particulate odor control material in an amount ranging from 5.0% to 45%, and preferably about 25% by weight, of the total weight of the particles. The sodium bicarbonate promotes skin wellness by controlling the pH levels of the fragrance oil, starch and human waste. A small amount of flow agent is also contained in the particulate odor control material. The odor control composition is indicated to be used in a disposable absorbent article for absorbing and containing body fluids, comprising an absorbent core and an odor control powder, both located between a fluid pervious cover sheet and a fluid impervious or hydrophobic backing. The odor control powder is substantially unscented when in an initial dry condition, before being wetted, and is capable of releasing a mild fragrance when wetted. The composition is not indicated to be usable in an animal litter.

U.S. Pat. Nos. 6,375,983 and 6,558,706 to Kantor, et al. disclose microencapsulated fragrances and method for preparation. This encapsulated fragrance has a microcapsule from which the fragrance is controlled, and released by exposing the encapsulated fragrance to a solution of a predetermined pH. The encapsulant for the microcapsule is a copolymer of acrylic acid monomer and a one ethylenically unsaturated polymerizable monomer. The copolymer further comprises a pH sensitive carboxyl group or an amine group. The microcapsule encapsulant dissolves when it contacts a solution of appropriate pH. This encapsulated fragrance is not indicated to contain cellulose material.

U.S. Pat. Nos. 6,638,591 and 6,902,817 to Bowen, et al. discloses membrane permeable to aromatic products. This multilayer structure with improved permeation for atmospheric diffusion of aromatic products has a structure with a first permeable layer of a blend of very low density polyethylene and low density polyethylene, a second permeable layer of low density polyethylene, a third permeable layer of a blend of very low density polyethylene and low density polyethylene, a fourth permeable layer of a material selected from a blend of low density polyethylene and a modified polyolefin and a release layer comprising ethylene vinyl alcohol copolymer. The multilayer wall structure of a close extruded cell releases aromatic compound at a slow rate.

U.S. Pat. Nos. 6,500,463 and 7,201,923 to Van Lengerich discloses encapsulation of sensitive (liquid) components into a matrix to obtain discrete shelf-stable particles. A liquid encapsulant component, which contains an active, sensitive encapsulant, such as a live microorganism or an enzyme dissolved or dispersed in a liquid plasticizer is admixed with a plasticizable matrix material. The matrix material is plasticizable by the liquid plasticizer and the encapsulation of the active encapsulant is accomplished at a low temperature and under low shear conditions. Release of an active component from the matrix may be delayed or controlled over time so that the active component is delivered when and where it is needed to perform its intended function. Controlled release, discrete, solid particles which contain an encapsulated and/or embedded component such as a heat sensitive or readily oxidizable pharmaceutically, biologically, or nutritionally active component are continuously produced without substantial destruction of the matrix material or encapsulant.

U.S. Pat. No. 7,235,261 to Smith, et al. discloses a controlled release encapsulation. The controlled release encapsulated dry powder is formed by an emulsion having a fully hydrolyzed polyvinyl alcohol polymer, a hydrophobic silica, a modified corn starch, and a fragrance oil. The fragrance oil is emulsified in water and spray dried to evaporate the water obtaining the encapsulated dry powder. The dry powder with encapsulated fragrance oil provides controlled release of the fragrance, presumably due to cracks and irregularities present in encapsulation wall.

U.S. Pat. No. 7,431,986 to Van Lengerich et al. discloses encapsulation of sensitive components using pre-emulsification. A stabilized emulsion is employed to produce shelf stable, controlled release, discrete, solid particles or pellets, which contain an encapsulated and/or embedded component, such as a readily oxidizable component, such as omega-3 fatty acids or fragrances. An oil encapsulant component which contains an active, sensitive encapsulant, dissolved and/or dispersed in an oil is admixed with an aqueous component and a film-forming component to form an emulsion. The emulsion is stabilized by subjecting it to homogenization. The pellets are produced by first reducing the water content of the stabilized emulsion so that the film-forming component forms a film around the oil droplets and encapsulates the encapsulant. The encapsulated article does not contain cellulosic material.

There remains a need in the art for a fragrant pellet that has a high fragrance capacity, releases fragrance at an adequate rate to freshen the environment of a drawer, a cabinet, a trashcan or a litter box cat or animal litter composition Also needed in the art is a fragrant pellet that does not overpower the environment with the fragrant scent; but at the same time depleted the fragrant scent incorporated within the fragrant pellet.

### SUMMARY OF THE INVENTION

The present invention provides fragrant compacted pellets or granules that have a cellulosic material such as peat moss and activated carbon in combination with a suitable binder, and which are compacted to form pellets or granules. The pellet is sprayed with or soaked in fragrant oil. This results in the swelling of the cellulosic material such as peat moss due to the inherently sponge-like texture of the compacted pellets or granules, which have a multitude of capillary passages within the pellet or granule. During use, the activated carbon present within the fragrant pellet or granule has a large surface area. As a result, the fragrant oil evaporates, providing adequate continuous scent release in the area surrounding the fragrant pellet. The cellulosic fibers of the fragrant pellet have fragrant oil retained and soaked therewithin. As such, the fragrant oil intimately contacts the activated carbon pellets. A fresh supply of fragrant oils is provided to the activated carbon pellets, preventing the exhaustion of fragrant scent. As a result, the fragrant pellets provide a slow consistent release of fragrance for a period well over several months.

Generally stated, the fragrance-scented pellets or granules have liquid fragrance oil infiltrated within a previously compacted cellulosic pellet or granule comprising activated carbon and appropriate binder. The cellulosic pellet or granule comprises cellulose material selected from peat moss, paper waste, saw dust, plant tissue or other natural cellulosic materials. The preferred cellulosic material is peat moss which absorbs the fragrance oil and swell readily. Other cellulosic plant fibers may need to be treated in pressurized steam or treated with alkali to achieve similar absorption efficiency. The overall surface area available for the evaporation of fragrance from the fragrance oil absorbed within a swollen cellulosic fiber compacted pellet or granule is generally small due to the availability only a small surface area. The compacted pellets or granules contain uniformly distributed activated carbon typically in the range of 10 to 50% by weight of the pellet. These micropores of activated carbon provide superb conditions for adsorption to occur, since adsorbing material can interact with many surfaces simultaneously. Thus the fragrance oil infiltrated into the compacted pellet or granules find their way into the micropores within the activated carbon evaporating the vapor species of the fragrance, readily providing a high level of fragrance release to the local environment surrounding the compacted pellets or granules, yet having a level of fragrance that is not over powering. As the fragrance oil is consumed by evaporation, it is instantly replaced within the micropores of the activated carbon. This is due to the intimate contact between the swollen cellulosic fibers that are in close proximity with the activated carbon particles distributed nearly uniformly within the compacted pellets or granules. Since the swollen cellulosic fibers absorb fragrance oil and swell, an adequate amount of absorbed fragrance oil is present. The absorbed fragrance oil is well in excess of that which is lost by evaporation from the activated carbon. Hence, the usable life time of the fragrant compacted pellets or granules is substantially large, typically in the range of several months in ambient conditions. The lifetime of the compacted pellets or granules may be extended by enclosing in a sealed container during storage, wherein the fragrance vapor pressure within the sealed container reaches an equilibrium vapor pressure that prevents evaporation of the fragrance from the activated carbon surface.

Formation of compacted pellets or granules requires the addition of a binder. The binder may be selected from starch based compositions such as carboxymethyl cellulose, hydroxypropyl cellulose, methyl cellulose. Carboxymethyl cellulose (CMC) is a cellulose derivative with carboxymethyl groups (—CH2-COOH) bound to some of the hydroxyl groups of the glucopyranose monomers that make up the cellulose backbone. Hydroxypropyl cellulose (HPC) is a derivative of cellulose with both water solubility and organic solubility. HPC is an ether of cellulose in which some of the hydroxyl groups in the repeating glucose units have been hydroxypropylated forming —OCH2CH(OH)CH3 groups. Methyl cellulose (or methylcellulose) is a chemical compound derived from cellulose. It is a hydrophilic white powder in pure form and dissolves in cold (but not in hot) water, forming a clear viscous solution or gel. The binder may also include plant based cellulosic materials including tapioca starch, xanthan gum, guar gum, guar gum derivatives, and karaya gum.

The process of manufacturing the compacted pellets or granules involves the following steps. The cellulosic material is mixed with 10 to 50 weight percent of activated carbon and binder is added to the mixture in quantities ranging from 2 to 10 weight percent. The mixture is thoroughly blended using an industrial grade Z blade mixture so that the cellulosic material, activated carbon and binder are intimately mixed. Pellets are formed by pressing the mixture within an enclosed die and applying adequate pressure typically in the range of 5 to 200 psi to compact the pellet. The mixture may be rolled between two rolls each having textured impressions so that granules are formed as the mixture passes through the space between the rolls. If the binder composition included water or solvent, the pressed pellets may be dried at room temperature or at an elevated temperature. At this stage, the compacted pellets or granules do not have any fragrance.

In a first embodiment, the compacted pellets or granules are immersed or sprayed with the fragrant oil. Due to the swelling properties of the cellulosic material and microporous nature of the activated carbon, the pellets or granules swell rapidly absorbing the fragrant oil. Experiments have shown that the compacted pellets with cellulosing fibers such as peat moss absorb only 5 weight percent of fragrant oil in the absence of activated carbon addition. In a similar manner, compacted pellets with only activated carbon also absorb only 5 weight percent of fragrant oil. When cellulosic material such as peat moss is present in combination with activated carbon, the fragrance oil in this microcapsule is mixed with an unscented litter, the fragrant oil absorption is typically in the range of 23-27 weight percent. The exact theory for this synergistic effect is not well understood. However, this results in a large reservoir of fragrant oil in the cellulosic material, such as peat moss, that can deliver the fragrant oil to the activated carbon. This is because the activated carbon evaporates the fragrance oil at a reasonable rate, due to large surface area of the activated carbon present. Due to the large reservoir of fragrant oil within the cellulosic material the fragrant compacted pellets or granules have a useful life of several months. Preferably, the fragrant compacted pellets or granules are marketed in a sealed container that reduces or prevents the evaporation of the fragrant oil from the activated carbon component of the fragrant compacted pellets or granules.

In a second embodiment, the compacted pellets or granules are marketed separately from the fragrant oil and the user adds the fragrant oil to the compacted pellets or granules and shakes the mixture until all the fragrant oil is absorbed. This procedure clearly results in a much longer lifetime of fragrant compacted pellets or granules as compared to pre-mixed version of the product.

The fragrant compacted pellets or granules may be sprinkled in a drawer or cabinet to provide a long lasting, non-overpowering fragrant scent. The fragrant compacted pellets or granules may be added to a trashcan along with trash at periodic intervals as trash is being accumulated. This provides a fragrant trash scent when the user opens the lid of the trashcan to add additional trash. The fragrant compacted granules may be added to unscented conventional animal litter such as cat litter to provide fragrance that is always being slowly released without overpowering a closed environment with fragrance. The fragrance essentially masks litter odor, especially cat litter odor.

The fragrance oil used may be selected from a number of natural or synthetic fragrances as well as odor masking compounds. Anti-microbial agents may also be included in combination with the fragrance oil, especially when fragrant compacted pellets or granules are made for trashcan use or animal litter use.

### BRIEF DESCRIPTION OF THE DRAWINGS

The invention will be more fully understood and further advantages will become apparent when reference is had to the following detailed description of the preferred embodiments of the invention and the accompanying drawing, in which:

FIG. 1 is a schematic diagram showing a fragrant compacted pellet wherein a swollen pellet with fragrant oil feeds the micropores of activated carbon;

FIG. 2 is a schematic diagram showing swollen fragrant compacted granules with fragrant oil feeding micropores of activated carbon;

FIG. 3 is a diagrammatic representation of the process of manufacturing a fragrant compacted pellet; and

FIG. 4 is a diagrammatic representation of the process of manufacturing a fragrant compacted granule for use in a litter box as an additive or in a drawer or cabinet for continued fragrance release;

FIG. 5 is a schematic diagram of the use of fragrant compacted granules in a trashcan; and

FIG. 6 is a schematic diagram of the use of fragrant compacted granules added to a cat littler box.

### DETAILED DESCRIPTION OF THE INVENTION

This invention relates to fragrant compacted pellets or granules that release fragrance continuously and at a consistent rate without overpowering the ambient with excess fragrance. The compacted pellets or granules have a combination of a cellulosic material such as peat moss and activated carbon both held together with a suitable binder. When the compacted pellets or granules are sprayed with or soaked in a fragrant oil, the cellulosic material such as peat moss swells due to the inherently sponge like texture of the compacted pellets or granules that has a multitude of capillary passages within the pellet or granule. The activated carbon present within the fragrant compacted pellet or granule has a large surface area resulting in evaporation of the fragrant oil providing continuous yet adequate scent release in the area surrounding the fragrant pellet or granule. The cellulosic fibers of the fragrant pellet has fragrant oil retained and soaked there within which intimately contacts the activated carbon pellets, providing a fresh supply of fragrant oils to the activated carbon pellets that prevents the evaporative exhaustion of fragrant scent. As a result, the fragrant pellets provide a slow consistent release of fragrance for a period well over several months.

The fragrant pellets or granules may be used as stand-alone pellets or granulates that may be sprinkled in drawers or cabinets. The pellets may be added to trash in a trashcan preventing malodor release. The fragrant granules may be added to pet litter to eliminate malodor. Since the fragrant pellet or granules only release pleasant fragrant scent at a rate according to the available surface area of the activated carbon, the scent is not released too rapidly to release an overpowering scent, yet at the same time it is sufficient to suppress the malodor.

Generally stated, the fragrance-scented pellets or granules have liquid fragrance oil infiltrated within a previously compacted cellulosic pellet or granule comprising activated carbon and appropriate binder. The cellulosic pellet comprises cellulose material that may be selected from a number of cellulosic fibers including peat moss, paper waste, plant tissue and other materials. The preferred cellulosic material is peat moss, which has already been sufficiently degraded, and as a result absorbs the fragrance oil readily and swells rapidly. Other cellulosic plant fibers may need to be treated in pressurized steam or treated with alkali to achieve similar absorption efficiency. The overall surface area available for the evaporation of fragrance from the fragrance oil absorbed within a swollen cellulosic fiber compacted pellet or granule is generally small due to the availability only small surface area. Thus, the swollen cellulose material only acts as a large reservoir of fragrant oil and does not evaporate the fragrance to the ambient readily.

The compacted pellets or granules also contain uniformly distributed activated carbon typically in the range of 10 to 50% by weight of the pellet. A gram of activated carbon has a surface area well in excess of 500 m², typically greater than 1500 m² range. Under an electron microscope, the high surface-area structures of activated carbon are revealed. Individual particles are intensely convoluted and display various kinds of porosity; there may be many areas where flat surfaces of graphite-like material run parallel to each other, separated by only a few nanometers or so. These micropores provide superb conditions for adsorption to occur, since adsorbing material can interact with many surfaces simultaneously. Thus the fragrance oil infiltrated into the compacted pellet or granules finds its way into the micropores within the activated carbon. The vapor species of the fragrance evaporates readily, providing a high level of fragrance release within the local environment that surrounds the compacted pellets or granules, without triggering a level of fragrance that is over powering. As the fragrance oil is consumed by evaporation from the activated carbon surface, it is instantly replaced within the micropores of the activated carbon from the swollen cellulosic fibers that are in close proximity with the activated carbon particles. Since the swollen cellulosic fibers absorb fragrance oil and swell, an adequate amount of absorbed fragrance oil is present that is well in excess of that which is lost by evaporation from the activated carbon. The usable life time of the fragrant compacted pellets or granules is substantially large, typically in the range of several months in ambient conditions. The lifetime of the compacted pellets or granules may be extended by enclosing them in a sealed container during storage. In such cases, the fragrance vapor pressure within the sealed container reaches an equilibrium vapor pressure, preventing evaporation of the fragrance from the activated carbon surface.

Formation of compacted pellets or granules requires the addition of a binder. The binder may be selected from starch based compositions such as carboxymethyl cellulose, hydroxypropyl cellulose, methyl cellulose. Carboxymethyl cellulose (CMC) is a cellulose derivative with carboxymethyl groups (—CH2-COOH) bound to some of the hydroxyl groups of the glucopyranose monomers that make up the cellulose backbone. Hydroxypropyl cellulose (HPC) is a derivative of cellulose with both water solubility and organic solubility. HPC is an ether of cellulose in which some of the hydroxyl groups in the repeating glucose units have been hydroxypropylated forming —OCH2CH(OH)CH3 groups. Methyl cellulose (or methylcellulose) is a chemical compound derived from cellulose. It is a hydrophilic white powder in pure form and dissolves in cold (but not in hot) water, forming a clear viscous solution or gel. The binder may also include plant based cellulosic materials including tapioca starch, xanthan gum, guar gum, guar gum derivatives, and karaya gum.

The process of manufacturing the compacted pellets or granules involves the following steps. The cellulosic material is mixed with 10 to 50 wt percent of activated carbon. Binder is added to the mixture in quantities ranging from 2 to 10 weight percent. The mixture is thoroughly blended using an industrial grade Z blade mixture so that the cellulosic material, activated carbon and binder are intimately mixed. Pellets are formed by pressing the mixture within an enclosed die and applying adequate pressure typically in the range of 5 to 200 psi to compact the pellet. The mixture may be rolled between two rolls each having a textured impressions so that granules are formed as the mixture passes through the space between the rolls. If the binder composition included water or solvent, the pressed pellets may be dried at an elevated temperature. At this stage, the compacted pellets or granules do not have any fragrance.

In a first embodiment, the compacted pellets or granules are immersed or sprayed with the fragrant oil. Due to the swelling properties of the cellulosic material and microporous nature of the activated carbon, the pellets or granules swell rapidly absorbing the fragrant oil. Experiments have shown that the compacted pellets with cellulosing fibers such as peat moss absorb only 5 weight percent of fragrant oil in the absence of activated carbon addition. In a similar manner, compacted pellets with only activated carbon also absorb only 5 weight percent of fragrant oil. When cellulosic material such as peat moss is present in combination with activated carbon, the fragrance oil in this microcapsule is mixed with an unscented litter. The fragrant oil absorption in such a case is typically in the range of 23-27 weight percent. The exact theory for this synergistic effect is not well understood. However, this results in a large reservoir of fragrant oil in the cellulosic material such as peat moss that can deliver the fragrant oil to the activated carbon. As such the fragrance oil evaporates at a reasonable rate due to large surface area of the activated carbon present. Due to the large reservoir of fragrant oil within the cellulosic material the fragrant compacted pellets or granules have a useful life of several months. Preferably, the fragrant compacted pellets or granules are marketed in a sealed container that reduces or prevents the evaporation of the fragrant oil from the activated carbon component of the fragrant compacted pellets or granules.

In a second embodiment, the compacted pellets or granules are marketed separately from the fragrant oil and the user adds the fragrant oil to the compacted pellets or granules and shakes the mixture until all the fragrant oil is absorbed. This procedure clearly results in a much longer lifetime of fragrant compacted pellets or granules as compared to pre-mixed version of the product.

The fragrant compacted pellets or granules may be sprinkled in a drawer or cabinet to provide a long lasting, non-overpowering fragrant scent. The fragrant compacted pellets may be added to a trashcan along with trash at periodic intervals as trash is being accumulated. This provides a fragrant trash scent when the user opens the lid of the trash can and adds new trash. The fragrant compacted granules may be added to unscented conventional animal litter such as cat litter to provide fragrance that is always being slowly released without overpowering a closed environment with fragrance. The fragrance essentially masks litter odor, especially cat litter odor.

The fragrance oil used may be selected from a number of natural or synthetic fragrances as well as odor masking compounds. Anti-microbial agents may also be included in combination with the fragrance oil, especially when fragrant compacted pellets or granules are made for trashcan use or animal litter use. The fragrance may be an odor neutralizing or masking compound or a pleasant smelling fragrance. A typical odor neutralizing or masking compound is lauryl methacrylate (sold under trade name METAZENE by Pestco Company). The masking compound is dissolved in acetone, a non-aqueous volatile carrier. Representative examples of fragrance components generally include, but are not limited to: volatile phenolic substances (such as iso-amyl salicylate, benzyl salicylate, and thyme oil red); essence oils (such as geranium oil, patchouli oil, and petitgrain oil); citrus oils; extracts and resins (such as benzoin siam resinold and opoponax resinold); “synthetic” oils such as Bergamot 37 and 430, Geranium 76 and Pomeransol 314, and Powder Mask CE-32907); aldehydes and ketones (such as beta-methyl naphthyl ketone, p-tert-butyl-a-methyl hydrocinnamic aldehyde and p-tert-amyl cyclohexanone); polycyclic compounds (such as Coumarin and beta-naphthyl methyl ether); esters (such as diethyl phthalate, phenylethyl phenylacetate). Fragrances also include esters and essential oils derived from floral materials and fruits, citrus oils, absolutes, aldehydes, etc. and alcohols (such as dimyrcetol, phenylethyl alcohol and tetrahydromuguol). Other fragrances include Cherry, Bonsai, Watermelon, Apple, Almond blend, Gamma, Cinnamon, Orange, Lemon, Eucalyptus, Honey Suckle, Citrus Orange, Ambient Neutralizer and Pine Oil. Generally the fragrances are dissolved in aqueous or non-aqueous carrier and the microcapsule cell wall is provided by well known means.

The following examples are provided to more completely describe the properties of the present invention. The specific techniques, conditions, materials, proportions and reported data set forth to illustrate the principles and practice of the invention are exemplary only and should not be construed as limiting the scope of the invention.

### Example 1

ARA fragrant compacted pellet is made by mixing 100 grams of peat moss with 40 grams of activated carbon and 5 grams of methyl cellulose and mixed thoroughly in a Z blade mixer. 5 to 10 cc of cold water is added to facilitate mixing of the ingredients. The mixture is pressed in a compression press with a die set to form a pressed tablet using a compression pressure of 50 psi. The compacted pellet is removed from the die set and is dried at 80° C. to remove the water content. Fragrance oil of the lemon scent is sprayed on the compacted pellet resulting in pellet swelling and fragrance oil absorption. The fragrant pellet is used in an automobile ash tray and maintained pleasant fragrance for well over one month.

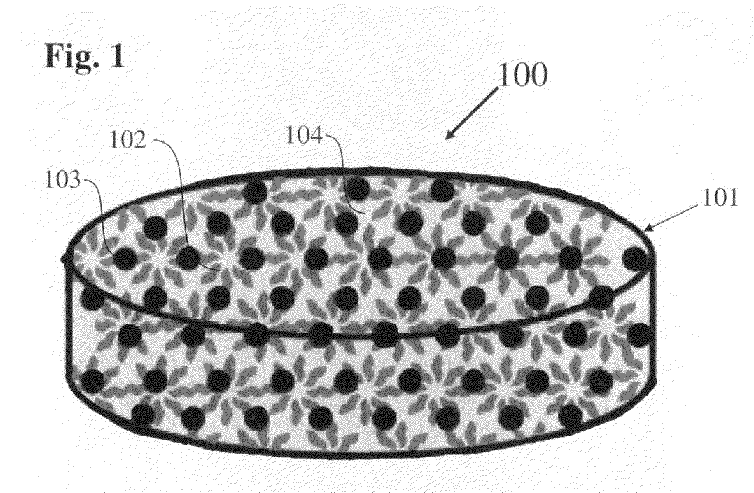

FIG. 1 illustrates at 100 a schematic diagram showing a fragrant compacted pellet wherein a swollen pellet 101 with fragrant oil feeds the micropores within activated carbon 103. The cellulosic material forms a matrix 102, which swells when the fragrant oil is absorbed and this abundant quantity of fragrant oil absorbed is shown at 104. The fragrant oil evaporates from the micropores of activated carbon 103 that has high surface area and this fragrance evaporation is maintained at a steady rate providing a pleasant fragrance in the environment surrounding the fragrant compacted pellet 101. The presence of an abundant quantity of fragrant oil in the cellulosic material feeds the micropores within activated carbon 103.

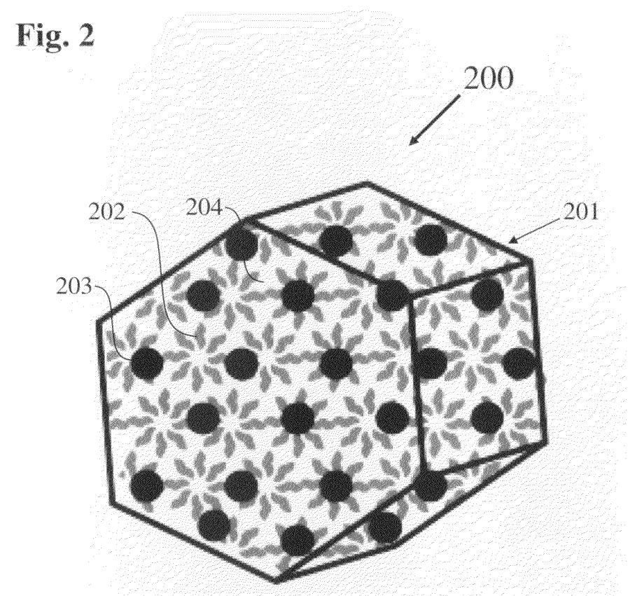

Referring to FIG. 2, there is shown generally at 200 a schematic diagram of a fragrant compacted granule, wherein a swollen pellet 201 with fragrant oil feeds the micropores within activated carbon 203. The cellulosic material forms a matrix 202, which swells when the fragrant oil is absorbed and this abundant quantity of fragrant oil absorbed is shown at 204. The fragrant oil evaporates from the micropores of activated carbon 203 that have high surface area, and this fragrance evaporation is maintained at a steady rate, providing a pleasant fragrance in the environment surrounding the fragrant compacted granule 201. The presence of an abundant quantity of fragrant oil in the cellulosic material feeds the micropores within activated carbon 203. The granules 201 shown are generally smaller in linear dimension, typically in the range of 0.5 mm to 6 mm as compared to that of pellets 101 shown in FIG. 1. Fragrant compacted pellets have a typical linear dimension in the range of 8 mm to 25 mm.

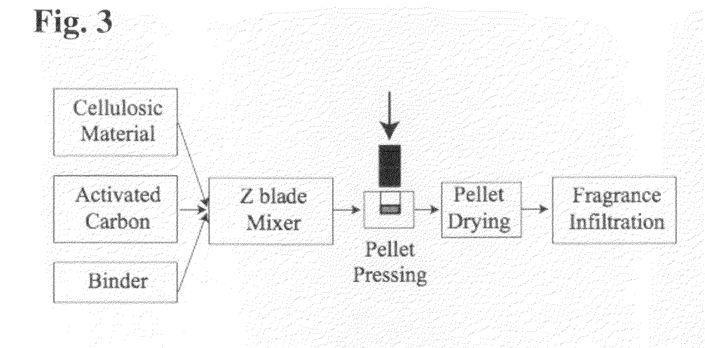

FIG. 3 is a diagrammatic representation of the process of manufacturing a fragrant compacted pellet for use in a trashcan or cabinets. The process involves selecting cellulosic material, activated carbon typically in the range of 10 to 50 weight percent, and a binder typically in the range of 2 to weight percent. These ingredients are thoroughly mixed in a mixture, which may be a Z blade mixture. The Z blades creates shear in the material and mixes the ingredients thoroughly, uniformly distributing the activated carbon and binder. Water or solvent may be added to the mixture to assist the mixing process. The mixture is fed in measured quantities into a die set that presses the compacted pellets under pressure, typically in the range of 5 to 200 psi. The molded compacted pellet is removed from the die set and dried to remove water or any solvent used to assist mixing. The pellet is free from fragrance and is in the unswelled compacted state. In the first embodiment, the compacted pellet is immersed in or sprayed with fragrant oil, which results in the swelling of the compacted pellet and absorption of the fragrant oil. In the second embodiment, the user adds the fragrant oil to the compacted pellet immediately before use.

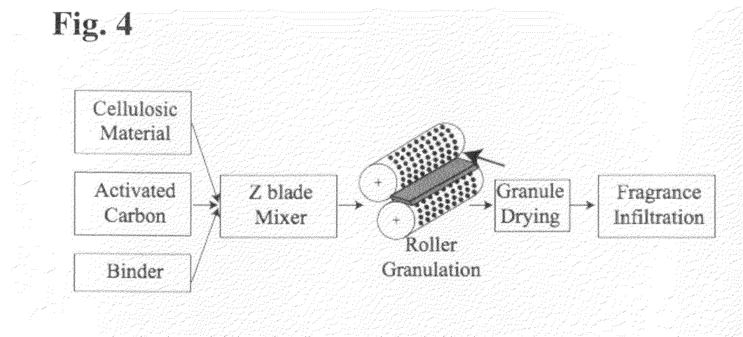

FIG. 4 is a diagrammatic representation of the process of manufacturing a fragrant compacted granule for use in litter boxes or cabinets. The process involves selecting cellulosic material, activated carbon typically in the range of 10 to 50 weight percent, and a binder typically in the range of 2 to weight percent. These ingredients are thoroughly mixed in a mixture, which may be a Z blade mixture. The Z blades create shear in the material and mix the ingredients thoroughly, uniformly distributing the activated carbon and binder. Water or solvent may be added to the mixture to assist the mixing process. The mixture is fed in the form of a sheet between two rollers. The rollers have impressions that produce granules as the sheet exits the space between the rollers. The molded, compacted granules are removed and dried to remove water or any solvent used to assist mixing. The granules are free from fragrance and are in the unswelled compacted state. In the first embodiment, the compacted granules are immersed in or sprayed with fragrant oil, which results in the swelling of the compacted granules and absorption of the fragrant oil. In the second embodiment, the user adds the fragrant oil to the compacted granules immediately before use.

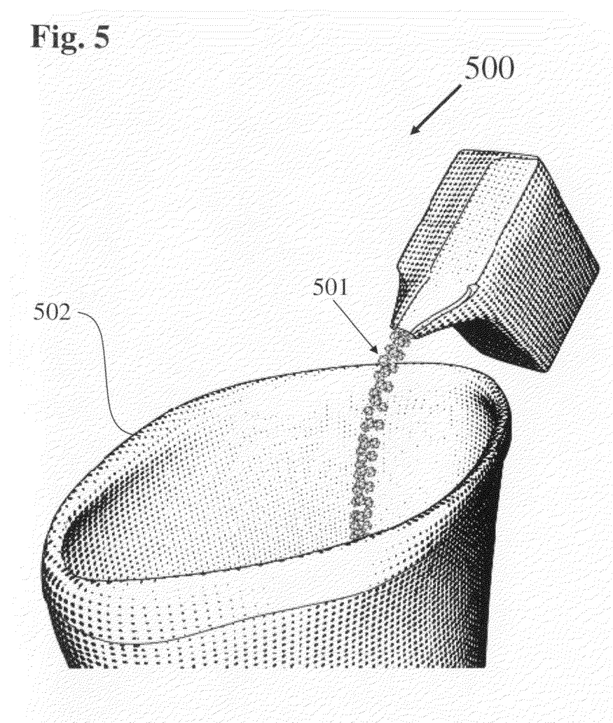

FIG. 5 is a schematic diagram of the use of fragrant compacted granules in a trashcan. The fragrant compacted granules are sprinkled into the interior of a trashcan from a container as shown. The presence of the fragrant compacted granules near the top most portion over trash within the trashcan releases fragrance, masking the malodor of trash.

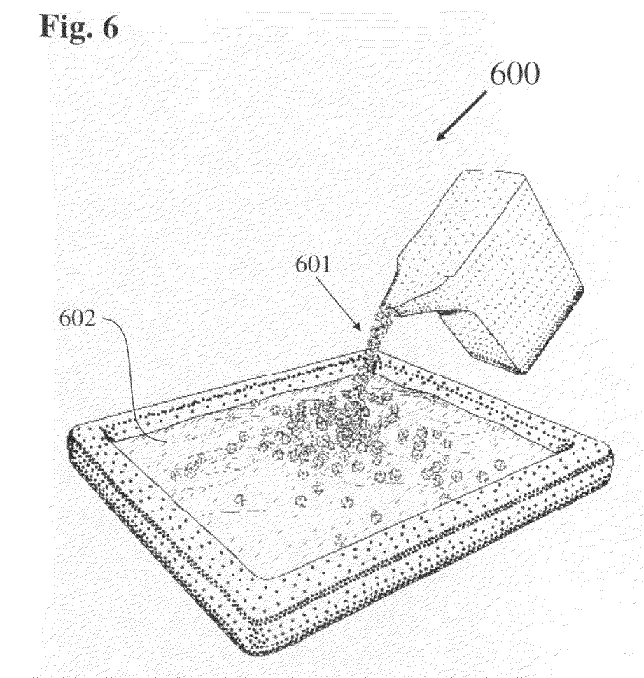

FIG. 6 is a schematic diagram of the use of fragrant compacted granules in a cat litter box. The fragrant compacted granules are sprinkled into the interior of a cat litter box from a container, as shown. The presence of the fragrant compacted granules within the litter box releases fragrance, masking the malodor of cat litter.

Significant advantages are realized by practice of the present invention. The key components of the odor control additive for animal litter include, in combination, the features set forth below;
1. an odor control fragrance additive comprising fragrant compacted pellets or granules saturated with fragrant oil;
2. said compacted pellets or granules comprising a compacted mixture of cellulosic material, activated carbon and a binder;
3. said cellulosic material being selected from peat moss, paper waste or plant based material and combination thereof;
4. said cellulosic material in compacted pellets or granules swelling and absorbing said fragrant oil;
5. said activated carbon having micropores with a large surface area adapted for steady fragrance release by evaporation of fragrant oil;
6. said swollen cellulosic material continuously feeding fragrant oil to said micropores of activated carbon, maintaining steady release of fragrance for a prolonged period of time;
whereby said fragrant compacted pellets or granules can be used in trashcans, litter boxes to suppress malodor and used in drawers or cabinets to release pleasant fragrance.

The process of manufacturing an odor control fragrance additive in the form of pellets or granules includes the steps set forth below:
1. selecting a cellulosic material including peat moss or paper waste or plant based material, or a combination thereof;
2. selecting activated carbon with micropores having high surface area in the 10 to 50 weight percent range, based on the weight of cellulosic material;
3. selecting a binder in the 2 to 10 weight percent range based on the weight of cellulosic material;
4. mixing cellulosic material, activated carbon and binder thoroughly to provide uniform distribution within a mixture;
5. compacting said mixture in a press under pressure to form compacted pellets;
6. compacting said mixture between textured rotating rolls to form compacted granules;
7. selecting a liquid fragrance oil with a fragrance vapor pressure greater than one atmosphere;
8. infiltrating said fragrant oil into said compacted pellets or granules forming fragrant compacted pellets or granules;
9. packaging for sale said fragrant compacted pellets or granules in sealed containers for use in trashcans, litter boxes or as stand-alone fragrant pellets or granules;
10. packaging for sale said compacted pellets or granules and fragrant oil, so that said pellets or granules and said fragrant oil can be separately mixed together by the user prior to deployment in trashcans, litter boxes or as stand-alone fragrant pellets or granules.

Having thus described the invention in rather full detail, it will be understood that such detail need not be strictly adhered to. For example, the compacted pellets or granules may be sold together with several fragrant oils allowing the user to select the desired fragrance in the fragrant compacted pellets or granules. A neutralizing agent can be used in addition to the fragrant oil to absorb and thereby irradiate odor otherwise produced by urine or defecation on the litter. Upon release, a natural bacteria producing enzyme contained by the neutralizing agent, reproduces itself continuously to eliminate all liter odor. These and additional changes and modifications may suggest themselves to one skilled in the art, all falling within the scope of the invention as defined by the subjoined claims.

What is claimed is:

1. An odor control fragrance additive, comprising:
a. fragrant compacted pellets or granules saturated with fragrant oil;
b. said compacted pellets or granules comprising a compacted mixture of cellulosic material, activated carbon and a binder;
c. said cellulosic material selected from peat moss, paper waste or plant based material, and combinations thereof;
d. said cellulosic material in compacted pellets or granules swelling and absorbing said fragrant oil;
e. said activated carbon having micropores with large surface area adapted for steady fragrance release by evaporation of fragrant oil;
f. said swollen cellulosic material continuously feeding fragrant oil to said micropores of activated carbon maintaining steady release of fragrance for a prolonged period of time;
whereby said fragrant compacted pellets or granules can be used in trashcans or litter boxes to suppress malodor, and used in drawers or cabinets to release pleasant fragrance.

2. The odor control fragrance additive as recited by claim 1, wherein said cellulosic material is peat moss.

3. The odor control fragrance additive as recited by claim 1, wherein said activated carbon has a surface area greater than 500 m² per gram.

4. The odor control fragrance additive as recited by claim 1, wherein said activated carbon has a surface area greater than 1500 m² per gram.

5. The odor control fragrance additive as recited by claim 1, wherein said activated carbon weight is in the range of 10 to 50 weight percent on the basis of the weight of cellulosic material.

6. The odor control fragrance additive as recited by claim 1, wherein said binder is selected from the group consisting of carboxymethyl cellulose, hydroxypropyl cellulose, methyl cellulose, tapioca starch, xanthan gum, guar gum, guar gum derivatives, karaya gum and combinations thereof.

7. The odor control fragrance additive as recited by claim 1, wherein said binder is in the range of 2 to 10 weight percent on the basis of the weight of cellulosic material.

8. The odor control fragrance additive as recited by claim 1, wherein said fragrant oil comprises an antimicrobial composition.

9. The odor control fragrance additive as recited by claim 1, wherein said fragrant oil comprises an odor neutralizing composition.

10. The odor control fragrance additive as recited by claim 1, wherein said fragrant compacted pellet has a linear dimension in the range of 8 mm to 25 mm.

11. The odor control fragrance additive as recited by claim 1, wherein said fragrant compacted granule has a linear dimension in the range of 0.5 mm to 6 mm.

12. A process for manufacturing an odor control fragrance additive, comprising the steps of;
a. selecting a cellulosic material including peat moss or paper waste or plant based material or a combination thereof;
b. selecting activated carbon with micropores having high surface area in the 10 to 50 weight percent range based on the weight of cellulosic material;
c. selecting a binder in the 2 to 10 weight percent range based on the weight of cellulosic material;
d. mixing cellulosic material, activated carbon and binder thoroughly to provide uniform distribution within a mixture;
e. compacting said mixture to form compacted pellets or compacted granules;
f. selecting a liquid fragrance oil with a fragrance vapor pressure greater than one atmosphere;
g. infiltrating said fragrant oil into said compacted pellets or granules to form fragrant compacted pellets or granules; and
h. packaging for sale said fragrant compacted pellets or granules.

13. A process for manufacturing an odor control fragrance additive as recited in claim 12, wherein said compacted pellets are formed by pressing said mixture in a die set under pressure.

14. A process for manufacturing an odor control fragrance additive as recited in claim 12, wherein said compacted granules are formed by passing said mixture between two counter rotating textured rolls.

15. A process for manufacturing an odor control fragrance additive as recited in claim 12, wherein said package is a sealed package comprising fragrant oil infiltrated compacted pellets or granules.

16. A process for manufacturing an odor control fragrance additive as recited in claim 12, wherein said package comprises separate fragrant oil and compacted pellets or granules to be intermixed by user prior to use.
